(19) United States
(12) Patent Application Publication
(10) Pub. No.: US 2025/0295838 A1
(43) Pub. Date: Sep. 25, 2025

(54) HYDROLYSIS-RESISTANT HYDROGELS AND METHODS OF TREATMENT USING SAME

(71) Applicant: Boston Scientific Scimed, Inc., Maple Grove, MN (US)

(72) Inventors: Heather Cirka, Hudson, MA (US); Yen-Hao Hsu, Shrewsbury, MA (US); Nishant Khattar, White Bear Township, MN (US); James M. Goddard, Pepperell, MA (US)

(73) Assignee: Boston Scientific Scimed, Inc., Maple Grove, MN (US)

(21) Appl. No.: 19/085,128

(22) Filed: Mar. 20, 2025

Related U.S. Application Data
(60) Provisional application No. 63/567,758, filed on Mar. 20, 2024.

(51) Int. Cl. A61L 27/52; A61L 27/18; C08J 3/075; C08J 3/24; C08L 71/02

(52) U.S. Cl. CPC A61L 27/52; A61L 27/18; C08J 3/075; C08J 3/24; C08L 71/02; A61L 2400/06

(57) ABSTRACT

In some aspects, the present disclosure provides systems for forming hydrogels that comprise (i) a reactive multi-arm polymer that comprises three or more polymer arms linked to a core region, each arm comprising a polyether segment and a reactive moiety comprising a cyclic imide carboxy-C₁-C₆-alkyl ester end group or each arm comprising a polyether segment and a reactive moiety comprising a C₂-C₆-isocyanoalkyl end group and (ii) a polyamino compound comprising at least two amino (—NH₂) groups, wherein the reactive multi-arm polymer and the polyamino compound react to form a crosslinked hydrogel that does not contain ester groups and has long-term stability in vivo. In some aspects, the present disclosure provides methods of treatment using such systems and hydrolysis-resistant crosslinked hydrogels formed from such systems.

### CROSS-REFERENCE TO RELATED APPLICATIONS

This application claims the benefit of U.S. Provisional Patent Application Ser. No. 63/567,758 filed on Mar. 20, 2024, the disclosure of which is incorporated herein by reference.

### FIELD

The present disclosure relates to hydrolysis-resistant hydrogels, to crosslinkable systems for forming such hydrolysis-resistant hydrogels, and to methods of treatment using such hydrolysis-resistant hydrogels.

### BACKGROUND

SpaceOAR®, a rapid crosslinking hydrogel that polymerizes in vivo within seconds, is based on a multi-arm polyethylene glycol (PEG) polymer with a polyol core functionalized with succinimidyl glutarate as reactive end groups which further react with trilysine to form crosslinks. This product has become a very successful, clinically-used biomaterial in prostate cancer therapy. A further improvement based on this structure is that a portion of the succinimidyl glutarate end groups have been replaced with 2,3,5-triiodobenzamide groups, providing radiopacity. This hydrogel, known by the trade name of SpaceOAR Vue®, is the radiopaque version of SpaceOAR® for prostate medical applications. Above a specific pH, the succinimidyl glutarate groups of SpaceOAR® and SpaceOAR Vue® will rapidly react with the trilysine crosslinker in vivo to form a hydrogel. The hydrogel breaks down in-vivo over the course of ca. 6-9 months. The breakdown occurs primarily through the hydrolysis of the ester linkages in the glutarate groups.

The present disclosure provides implantable hydrogel alternatives to SpaceOAR® and SpaceOAR Vue®, which are hydrolysis-resistant and have long-term stability, thereby expanding the range of medical applications for the hydrogels.

### SUMMARY

In some aspects, the present disclosure provides systems for forming hydrogels that comprise (i) a reactive multi-arm polymer that comprises three or more polymer arms linked to a core region, each arm comprising a polyether segment and a reactive moiety comprising a cyclic imide carboxy-C₁-C₅-alkyl ester end group or each arm comprising a polyether segment and a reactive moiety comprising a C₂-C₆-isocyanoalkyl end group and (ii) a polyamino compound comprising at least two amino (—NH₂) groups, wherein the reactive multi-arm polymer and the polyamino compound react to form a crosslinked hydrogel that does not contain ester groups and has long-term stability in vivo.

In some embodiments, the reactive moiety is a cyclic imide carboxy-C₁-C₅-alkyl ester group or a C₂-C₆-isocyanoalkyl group and the reactive moiety is directly bonded to the polyether segment.

In some embodiments, which may be used in conjunction with the above aspects and embodiments, the reactive multi-arm polymer is formed from a multi-arm polymer that comprises three or more polymer arms linked to a core region, each arm comprising a polyether segment and a C₂-C₆-hydroxyalkyl end group. In some of these embodiments, the reactive multi-arm polymer is formed by a process that comprises oxidizing the C₂-C₆-hydroxyalkyl end group to form a C₂-C₆-carboxyalkyl end group and reacting the C₂-C₆-carboxyalkyl end group with a N-hydroxy cyclic imide compound in an ester coupling reaction to form the cyclic imide carboxy-C₁-C₅-alkyl ester end group. In some of these embodiments, the reactive multi-arm polymer is formed from a process that comprises converting the C₂-C₆-hydroxyalkyl end group to a C₂-C₆-aminoalkyl end group and reacting the C₂-C₆-aminoalkyl end group with phosgene to form the C₂-C₆-isocyanoalkyl end group.

In some embodiments, which may be used in conjunction with the above aspects and embodiments, the polyether segment is a polyethylene oxide segment.

In some embodiments, which may be used in conjunction with the above aspects and embodiments, the C₂-C₆-isocyanoalkyl end group is an isocyanoethyl end group.

In some embodiments, which may be used in conjunction with the above aspects and embodiments, the cyclic imide carboxy-C₁-C₅-alkyl ester end group is a cyclic imide carboxymethyl ester end group. In particular embodiments, the cyclic imide carboxy-C₁-C₅-alkyl ester end group is a succinimide carboxymethyl ester end group.

In some embodiments, which may be used in conjunction with the above aspects and embodiments, each of the polyether segments contains between 10 and 1000 monomer residues.

In some embodiments, which may be used in conjunction with the above aspects and embodiments, the core region comprises a polyol residue.

In some embodiments, which may be used in conjunction with the above aspects and embodiments, the core region is an iodinated core region.

In some embodiments, which may be used in conjunction with the above aspects and embodiments, the polyamino compound comprises a plurality of basic amino acid residues.

In some embodiments, which may be used in conjunction with the above aspects and embodiments, the polyamino compound is an iodinated polyamino compound.

In some embodiments, which may be used in conjunction with the above aspects and embodiments, the system comprises a first composition that comprises the polyamino compound in a first container and a second composition that comprises the reactive multi-arm polymer in a second container. In some of these embodiments, the first container and second containers are independently selected from vials and syringe barrels. In some of these embodiments, the first container is a syringe barrel and the second container is a vial.

In some embodiments, which may be used in conjunction with the above aspects and embodiments, the system further comprises a delivery device. In some of these embodiments, the delivery device is a double barrel syringe.

In other aspects, the resent disclosure pertains to a hydrolysis-resistant crosslinked hydrogel produced by a system in accordance with any of the above aspects and embodiments.

In some embodiments, the hydrolysis-resistant crosslinked hydrogel has a radiopacity that is greater than 100 Hounsfield units (HU).

In some embodiments, which may be used in conjunction with the above aspects and embodiments, the hydrolysis-resistant crosslinked hydrogel is in particulate form, which may be delivered, for example, in a suspension from a single syringe barrel.

In some aspects, the present disclosure pertains to methods of treatment comprising administering to a subject a mixture that comprises (i) a reactive multi-arm polymer that comprises three or more polymer arms linked to a core region, each arm comprising a polyether segment and a reactive moiety comprising a cyclic imide carboxy-C₁-C₅-alkyl ester end group or each arm comprising a polyether segment and a reactive moiety comprising a C₂-C₆-isocyanoalkyl end group and (ii) a polyamino compound comprising at least two amino (—NH₂) groups, wherein the mixture is administered under conditions such that the polyamino compound and the reactive multi-arm polymer react to form a crosslinked hydrogel after administration and wherein the crosslinked hydrogel does not contain esters and has long-term stability in vivo.

In some embodiments, wherein the method comprises administering to the subject (a) a first fluid composition that comprises the polyamino compound and the reactive multi-arm polymer, wherein the reactive moiety comprises the cyclic imide carboxy-C₁-C₅-alkyl ester end group, and (b) a second fluid composition that comprises an accelerant that accelerates reaction of the polyamino compound and the reactive multi-arm polymer. In some of these embodiments, the first fluid composition and the second fluid composition are delivered using a double barrel syringe.

In some embodiments, wherein the method comprises administering to the subject (a) a first fluid composition that comprises the polyamino compound and a non-aqueous solvent and (b) a second fluid composition comprising the reactive multi-arm polymer and a non-aqueous solvent, wherein the reactive moiety comprises the C₂-C₆-isocyanoalkyl end group. In some of these embodiments, the first fluid composition and the second fluid composition are delivered using a double barrel syringe.

In some embodiments, which may be used in conjunction with the above aspects and embodiments, the mixture is injected into a bodily sphincter.

In some embodiments, which may be used in conjunction with the above aspects and embodiments, the mixture is injected into a urethral sphincter. In some of these embodiments, the mixture is injected by a transurethral route into a bladder neck of the subject through a urethral wall of the subject.

The above and other aspects, embodiments, features and benefits of the present disclosure will be readily apparent from the following detailed description.

### BRIEF DESCRIPTION OF THE DRAWINGS

FIG. 1 schematically illustrates the formation of a hydroxyl-terminated 8-arm-PEG having a tripentaerythritol residue core, in accordance with an embodiment of the present disclosure.

FIG. 2 schematically illustrates the formation of a hydroxyl-terminated 3-arm-PEG having a 1,3,5-triiodo-2,4,6-tris-hydroxymethylbenzene residue core, in accordance with an embodiment of the present disclosure.

FIG. 3 schematically illustrates the formation of a succinimide-carboxymethyl-ester-terminated multi-arm PEG, in accordance with an embodiment of the present disclosure.

FIG. 4 schematically illustrates the formation of an isocyanomethyl-terminated multi-arm PEG, in accordance with an embodiment of the present disclosure.

FIG. 5 schematically illustrates the formation of a crosslinked polymer network by reaction of trilysine with a succinimide-carboxymethyl-ester-terminated eight-arm PEG, in accordance with an embodiment of the present disclosure.

FIG. 6 schematically illustrates a delivery device, in accordance with an embodiment of the present disclosure.

FIGS. 7A and 7B schematically illustrate the injection of a crosslinked polymer network into a bladder neck of a patient, in accordance with an embodiment of the present disclosure.

FIG. 8 illustrates a delivery device, in accordance with another embodiment of the present disclosure.

### DETAILED DESCRIPTION

In some aspects of the present disclosure, hydrolysis-resistant hydrogels are provided that comprises a crosslinked reaction product of (a) a polyamino compound and (b) a reactive polymer comprising reactive moieties that are reactive with the amino groups of the polyamino compound, wherein the crosslinked reaction product does not contain esters or other readily hydrolysable groups that would lead to breakdown of the crosslinked reaction product upon implantation. Such hydrogels have long-term stability once implanted, opening up a variety of medical applications.

As used herein, a “hydrogel” is a crosslinked polymer that contains water or can absorb water but does not dissolve when placed in water. As used herein, an implanted hydrogel has “long-term stability” if it undergoes less than 25 wt % bioresorption, preferably less than 10 wt %, over the course of at least 5 years, preferably 10 years, after implantation in subject.

Reactive polymers for use in the present disclosure include reactive multi-arm polymers that comprise a plurality of polymer arms linked to a core region, wherein the polymer arms comprise a hydrophilic polymer segment. In some embodiments, a first end of the hydrophilic polymer segment is covalently linked to the core region and a reactive moiety is covalently linked to a second end (opposite end) of the hydrophilic polymer segment.

Reactive polymers in accordance with the present disclosure include polymers having from 3 to 100 arms, for example ranging anywhere from 3 to 4 to 5 to 6 to 7 to 8 to 10 to 12 to 15 to 20 to 25 to 50 to 75 to 100 arms.

Reactive moieties include moieties that comprise electrophilic groups and moieties that comprise isocyanate groups.

Electrophilic groups may be selected, for example, from cyclic imide ester

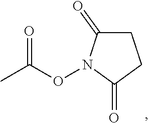

groups, such as succinimide ester groups, maleimide ester groups, glutarimide ester groups, diglycolimide ester groups, phthalimide ester groups, and bicyclo[2.2.1] hept-5-ene-2,3-dicarboxylic acid imide ester groups,

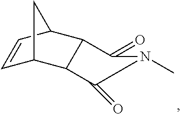

imidazole ester groups, imidazole carboxylate groups and benzotriazole ester groups, among other possibilities.

The electrophilic or isocyanate groups may be linked to the hydrophilic polymer segment through any suitable hydrolysis-resistant linking moiety, which may be selected, for example, from a linking moiety that comprises a C₁-C₆ alkyl group, a linking moiety that comprises an ether group, a linking moiety that comprises an amide group, a linking moiety that comprises a urethan group, a linking moiety that comprises a urea group, or a linking moiety that comprises a combination of two or more of the foregoing groups, among others.

In some embodiments, the cyclic imide ester groups are cyclic imide carboxy-C₁-C₆-alkyl ester groups, including succinimide carboxy-C₁-C₆-alkyl ester groups, maleimide carboxy-C₁-C₆-alkyl ester groups, glutarimide carboxy-C₁-C₆-alkyl ester groups, diglycolimide carboxy-C₁-C₆-alkyl ester groups, phthalimide carboxy-C₁-C₆-alkyl ester groups, and bicyclo[2.2.1] hept-5-ene-2,3-dicarboxylic acid imide carboxy-C₁-C₆-alkyl ester groups, among other possibilities. Specific embodiments described below include succinimide carboxymethyl ester groups, succinimide carboxyethyl ester groups and succinimide carboxypropyl ester groups.

In some embodiments, the cyclic imide carboxy-C₁-C₅-alkyl ester group is directly bonded to the hydrophilic polymer segment.

Hydrophilic polymer segments for the polymer arms can be selected from a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), and polar aprotic vinyl monomers (e.g. N-vinyl pyrrolidone, acrylamide, N-methyl acrylamide, N-isopropylacrylamide, dimethyl acrylamide, N-vinylimidazole, 4-vinylimidazole, sodium 4-vinylbenzenesulfonate, etc.).

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(N-isopropylacrylamide) segments, poly(dimethyl acrylamide) segments, poly(N-vinylimidazole) segments, poly(4-vinylimidazole) segments, and poly(sodium 4-vinylbenzenesulfonate) segments.

Polymer segments for use in the multi-arm polymers of the present disclosure typically contain between 10 and 1000 monomer units or more.

In certain embodiments, the core region comprises a residue of a non-iodinated or iodinated polyol comprising three or more hydroxyl groups, which is used to form the polymer arms. In certain beneficial embodiments, the core region comprises a residue of a polyol that contains from 3 to 100 hydroxyl groups, for example ranging anywhere from 3 to 4 to 5 to 6 to 7 to 8 to 9 to 10 to 11 to 12 to 15 to 20 to 25 to 50 to 75 to 100 hydroxy groups.

In some embodiments of the present disclosure, a non-iodinated or iodinated polyol such as one of those described below, among others, may be used as multi-functional initiator for polymer chain growth. For example, the non-iodinated or iodinated polyol may be used as an initiator for ring-opening polymerization of ethylene oxide to form polyethylene oxide (PEO) segments (also referred to a polyethylene glycol, or PEG, segments) at each of the hydroxyl groups of the polyol. The resulting hydroxyl-terminated PEG segments possess tunable hydrophilicity depending on the desired water-solubility of the resulting multi-arm polymer, for example, with increasing PEG segment length leading to increasing hydrophilicity.

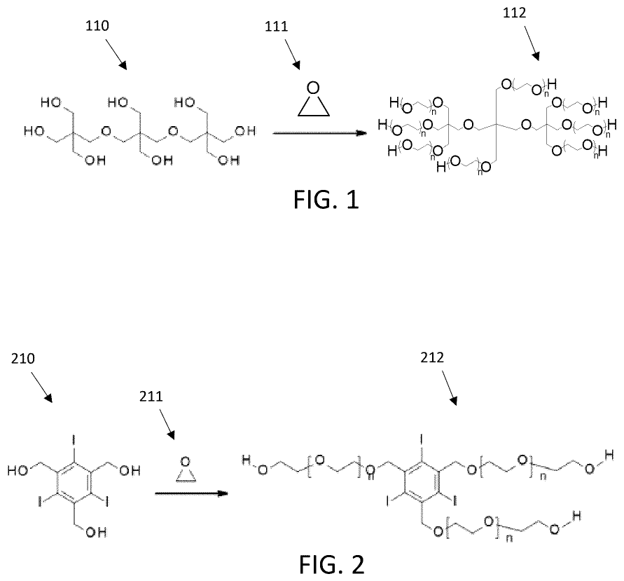

In a particular embodiment shown in FIG. 1, commercially available tripentaerythritol (110) (CAS #78-24-0) can be used as an octa-functional initiator, which undergoes ring-opening polymerization with ethylene oxide (111). The polymerization process leads to poly(ethylene oxide) (PEG) chain growth at each of the eight hydroxyl groups of the tripentaerythritol, forming a hydroxyl-terminated 8-arm-PEG (112) having a tripentaerythritol residue core. In FIG. 1, n is an integer representing the number of monomer units in each polymer segment shown.

The strategy shown in FIG. 1 is broadly applicable and can be used in conjunction with a range of polyols, including those described below. In a particular embodiment shown in FIG. 2, an iodinated polyol, specifically, 1,3,5-triiodo-2,4,6-tris-hydroxymethylbenzene (210), is used as an initiator, which undergoes ring-opening polymerization with ethylene oxide (211). The polymerization process leads to poly(ethylene oxide) (PEG) chain growth at each of the three hydroxyl groups of the 1,3,5-triiodo-2,4,6-tris-hydroxymethylbenzene (210). The resulting multi-arm polymer (212) contains three PEG arms that extend from a 1,3,5-triiodo-2,4,6-tris-hydroxymethylbenzene residue core. Each of the PEG arms has a terminal hydroxyl group. In FIG. 2, n is an integer representing the number of monomer units in each polymer segment shown.

Further illustrative non-iodinated and iodinated polyols for use in the present disclosure are described below. Such polyols may be used to form multi-arm polymeric polyols as described, for example, in FIGS. 1 and 2 above.

Non-iodinated polyols may be selected, for example, from sugars (monosaccharides, disaccharides, trisaccharides, etc.), sugar alcohols, calixarenes, cyclodextrins, polyhydroxylated polymers, catechins, flavanols, anthocyanins, stilbenes, and polyphenols, among others.

Non-iodinated polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols. Specific examples include methane triol, glycerol, trimethylolpropane, benzenetriol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, catechins, flavanols, anthocyanins, stilbenes, polyphenols, as well as polyhydroxy crown ethers, and polyhydroxyalkyl crown ethers. Illustrative polyols also include aromatic polyols including 1,1,1-tris(4′-hydroxyphenyl)alkanes, such as 1,1,1-tris(4-hydroxyphenyl) ethane, and 2,6-bis(hydroxyalkyl) cresols, among others.

Illustrative non-iodinated polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

Iodinated polyols are desirable where radiopacity is desired. Iodinated polyols include iodinated aromatic polyols, examples of which are compounds that comprise 3 or more hydroxyl groups, and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with two or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, ether groups, alkyl groups, and combinations thereof, among others.

Further iodinated polyols for use in the present disclosure, in addition to the 1,3,5-triiodo-2,4,6-tris-hydroxymethylbenzene described above, include iodinated polyols that are known for use as iodinated contrast agents, whose biocompatibility has been demonstrated to be reasonably well tolerated. Specific examples of iodinated polyols include commercially available 1,3,5-triiodo-2,4,6-trishydroxymethylbenzene (CAS #178814-33-0), iodixanol (CAS #92339 Nov. 2), iotrolan (CAS #79770-24-4), iohexol (CAS #66108-95-0), ioversol (CAS #87771-40-2), iopamidol (CAS #60166-93-0), iohexol impurity J (CAS #76801-93-9), and iopromide (CAS #73334 Jul. 3), among others.

Reactive multi-arm polymers in accordance with the present disclosure can be formed from hydroxyl-terminated precursor multi-arm polymers having arms that comprise one or more hydroxyalkyl end groups. In an initial step, the hydroxyalkyl end groups of a hydroxyl-terminated precursor multi-arm polymer can be oxidized to form a carboxylic-acid-terminated intermediate multi-arm polymer having arms that each comprise one or more carboxylic acid end groups. A reactive moiety may then be linked to the carboxylic-acid-terminated intermediate multi-arm polymer.

In some embodiments, an electrophilic moiety may be linked to the carboxylic-acid-terminated precursor polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1] hept-5-ene-2,3-dicarboxylic acid imide (HONB), etc.) may be reacted with the carboxylic-acid-terminated precursor polymer in the presence of a suitable coupling agent (e.g., a carbodiimide coupling agent, such as N,N′-dicyclohexylcarbodiimide (DCC), 1-ethyl-3-(3-dimethyl propyl) carbodiimide (EDC), N-hydroxybenzotriazole (HOBt), BOP reagent, and/or another coupling agent) to form a reactive cyclic imide ester (e.g., a succinimide ester group, a maleimide ester group, a glutarimide ester group, a phthalimide ester group, a diglycolimide ester group, a bicyclo[2.2.1] hept-5-ene-2,3-dicarboxylic acid imide ester group, etc.) that is linked to a hydrophilic polymer segment. In this way, a variety of reactive ester groups can be formed.

In a particular embodiment, an intermediate multi-arm polymer may be formed by oxidizing hydroxyalkyl end groups (e.g., C₂-C₆-hydroxyalkyl end groups) of a hydroxyl-terminated multi-arm precursor polymer with an oxidizing agent such as potassium dichromate (VI) or potassium permanganate, thereby forming carboxyalkyl end groups (e.g., C₂-C₆-carboxyalkyl end groups) at the sites previously occupied by the hydroxyalkyl end groups.

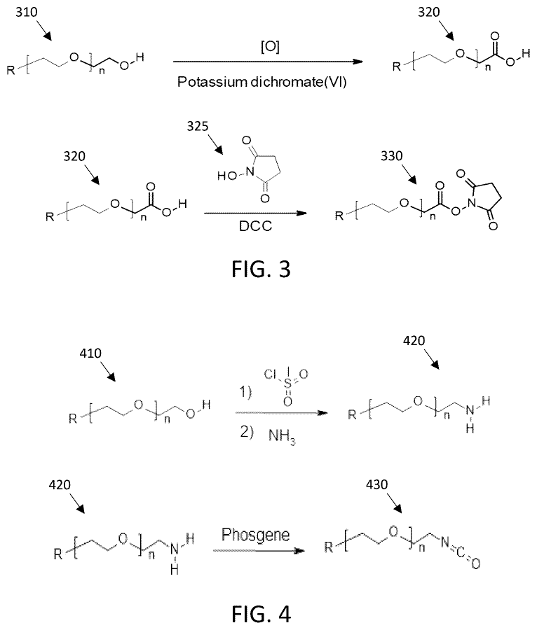

In one embodiment shown in FIG. 3, an intermediate multi-arm polymer, which comprises a core region and a plurality of polyethylene oxide (PEO) arms having carboxylic acid end groups is formed by oxidizing hydroxyalkyl end groups of a hydroxyl-terminated multi-arm polymer having a core region that comprises a polyol residue and multiple hydroxyl-terminated polyethylene oxide arms. In particular, a hydroxyl-terminated 8-arm PEG (310), where R comprises a tripentaerythritol or hexaglycerol polyol residue and n ranges from 30 to 140 (only one polymer arm is shown, and R represents the polyol residue and the remaining seven arms), is oxidized by reaction with potassium dichromate (VI) to create a carboxylic-acid-terminated 8-arm PEO (320). Then, in a subsequent step, the carboxylic-acid-terminated 8-arm PEO (320) is reacted with N-hydroxysuccinimide (325) in the presence of a coupling agent, specifically, N,N′-dicyclohexylcarbodiimide (DCC), to form a succinimide-ester-terminated 8-arm polymer (330) in which the succinimide ester groups are linked to the PEO segments through a methylene group, also referred to herein as a succinimide carboxymethyl ester group or a succinimidyl carboxymethyl ester group. In embodiments where a multi-arm polypropylene oxide is employed, an analogous product would be formed except that the succinimide ester groups will be linked to the polypropylene oxide segments through an ethylene group, also referred to herein as a succinimide carboxyethyl ester group or a succinimidyl carboxyethyl ester group. In embodiments where a multi-arm polytetramethylene oxide is employed, an analogous product would be formed except that the succinimide ester groups will be linked to the polytetramethylene oxide segments through a propylene (i.e., trimethylene) group, also referred to herein as a succinimide carboxypropyl ester group or a succinimidyl carboxypropyl ester group.

Reactive multi-arm polymers having reactive moieties that comprise isocyanate groups can also be formed from hydroxy-terminated precursor multi-arm polymers having arms that comprise one or more hydroxyl end groups.

In some embodiments, a multi-arm polymer having isocyanoalkyl end groups (e.g., C₂-C₆-isocyanoalkyl end groups) may be formed from a multi-arm polymer having hydroxyalkyl end groups (e.g., C₂-C₆-hydroxyalkyl end groups).

With reference to FIG. 4, a multi-arm polymer that comprises a core region and a plurality of polyethylene oxide (PEO) arms having isocyanate end groups is formed by reacting hydroxyalkyl end groups of a hydroxyl-terminated multi-arm polymer having a core region that comprises a polyol residue and multiple hydroxyl-terminated polyethylene oxide arms. More particularly, a hydroxyl-terminated 8-arm PEG (410) where R comprises tripentaerythritol or hexaglycerol polyol residue and n ranges from 30 to 140 (only one polymer arm is shown; the polyol residue and the remaining seven arms are represented by R), is first treated with methanesulfonyl chloride (CAS #124-63-0) to form an intermediate polymer in which hydroxyl groups of the iodixanol are converted into methanesulfonate groups, which methanesulfonate groups are then reacted with ammonia, followed by treatment with hydrochloric acid to convert the methanesulfonate groups to amino groups (in ammonium salt form), thereby forming an amino-terminated multi-arm PEO (420). Then, the amino-terminated multi-arm PEO (420) is reacted with phosgene to form an isocyanate-terminated multi-arm PEO (430). More generally, these reaction steps can be used to form isocyanoalkyl end groups (e.g., C₂-C₆-isocyanoalkyl end groups) from aminoalkyl end groups (e.g., C₂-C₆-aminoalkyl end groups) at the sites previously occupied by hydroxyalkyl end groups (e.g., C₂-C₆-hydroxyalkyl end groups) as indicated above.

As previously noted, in some aspects, the present disclosure provides hydrolysis-resistant crosslinked hydrogels that comprise a hydrolysis-resistant crosslinked reaction product of (a) a polyamino compound and (b) a reactive polymer comprising reactive moieties that are reactive with amino groups of the polyamino compound.

Such polyamino compounds include non-iodinated polyamino compounds and iodinated polyamino compounds.

Non-iodinated polyamino compounds suitable for use in the present disclosure include, for example, polyamines that contain at least two amino (—NH₂) groups (e.g., 2, 3, 4, 5, 6, 7, 8, 9, 10 or more amino groups in some embodiments). Non-iodinated polyamino compounds suitable for use in the present disclosure include those that comprise a plurality of —(CH₂)ₓ—NH₂ groups where x is 0, 1, 2, 3, 4, 5 or 6. Non-iodinated polyamino compounds suitable for use in the present disclosure include polyamino compounds that comprise basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.).

Further particular examples of non-iodinated polyamino compounds which may be used as the multifunctional compound include ethylenetriamine, diethylene triamine, hexamethylenetriiamine, di(heptamethylene) triamine, di(trimethylene)triamine, bis(hexamethylene)triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, and poly(allyl amine), among others.

Where additional radiopacity is desired in the crosslinked hydrogel, iodinated polyamine compounds may be employed.

In some embodiments, the iodinated polyamino compounds comprise a polyamino moiety that is linked to a carboxy-substituted iodinated moiety through an amide group. The iodinated polyamino compounds may comprise peptide oligomers that contain from 2 to 10 lysine and/or ornithine amino acid residues and one or more iodinated amino acid residues. The carboxy-substituted iodinated moieties may comprise iodinated amino acid residues.

Examples of iodinated amino acid residues include iodinated alpha-amino acid residues, iodinated beta-amino acid residues, iodinated gamma-amino acid residues and iodinated delta-amino acid residues. Examples of iodinated amino acid residues include amino acid residues that comprise an iodinated aromatic group. Examples of iodinated aromatic groups include iodo-phenyl groups and iodo-naphthyl groups. In particular embodiments, the iodinated amino acid residues include amino acid residues that comprise a hydroxy-iodo-aromatic group, such as a hydroxy-iodo-phenyl group or a hydroxy-iodo-naphthyl group. More particular examples of hydroxy-iodo-aromatic groups include hydroxy-iodo-phenyl groups selected from a mono-hydroxy-mono-iodo-phenyl group, a mono-hydroxy-di-iodo-phenyl group, a mono-hydroxy-tri-iodo-phenyl group, a mono-hydroxy-tetra-iodo-phenyl group, a di-hydroxy-mono-iodo-phenyl group, a di-hydroxy-di-iodo-phenyl group, a di-hydroxy-tri-iodo-phenyl group, a tri-hydroxy-mono-iodo-phenyl group, a tri-hydroxy-di-iodo-phenyl group, as previously indicated.

Specific examples of iodinated amino acid residues include residues of the following iodinated amino acids: iodo-phenylalanine,

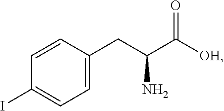 [SMILES: N[C@@H](Cc1ccc(I)cc1)C(=O)O]

which comprises a mono-iodo-phenyl group, monoiodotyrosine,

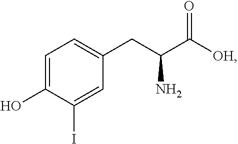 [SMILES: N[C@@H](Cc1ccc(O)c(I)c1)C(=O)O]

which comprises a mono-iodo-phenyl group, specifically, a mono-hydroxy-mono-iodo-phenyl group, diiodotyrosine,

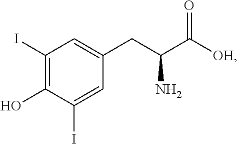 [SMILES: N[C@@H](Cc1cc(I)c(O)c(I)c1)C(=O)O]

which comprises a mono-hydroxy-di-iodo-phenyl group, diiodothyronine,

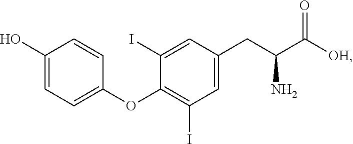 [SMILES: N[C@@H](Cc1cc(I)c(Oc2ccc(O)cc2)c(I)c1)C(=O)O]

which comprises a di-iodo-phenyl group and a hydroxy-phenyl group, triiodothyronine also known as T3,

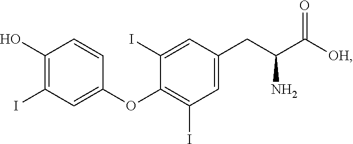 [SMILES: N[C@@H](Cc1cc(I)c(Oc2ccc(O)c(I)c2)c(I)c1)C(=O)O]

which comprises a di-iodo-phenyl group and a mono-hydroxy-mono-iodo-phenyl group, tetraiodothyronine also known as thyroxine or T4,

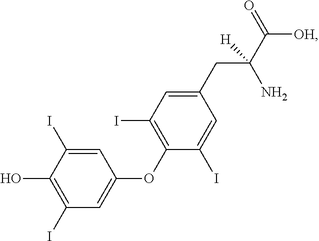 [SMILES: N[C@@H](Cc1cc(I)c(Oc2cc(I)c(O)c(I)c2)c(I)c1)C(=O)O]

which comprises a di-iodo-phenyl group and a mono-hydroxy-di-iodo-phenyl group, iodo-phenylalanine, and 6-iodo-L-DOPA, which comprises a di-hydroxy-mono-iodo-phenyl group, among others.

Iodinated polyamino compounds may be formed by an amide coupling reaction between (a) an iodinated amino acid derivative, for example, a C₁-C₅-alkyl ester of an iodinated amino acid, preferably a methyl ester of an iodinated amino acid, and (b) a carboxyl-substituted polyamino compound, selected for example, from these described below (after protection of the amino groups). Examples of such iodinated amino acid derivatives include C₁-C₅-alkyl esters of any of the preceding iodinated amino acids. After coupling, the protective groups on the residue of the carboxyl-substituted polyamino compound are removed and the C₁-C₅-alkyl ester may be converted into the corresponding carboxylic acid or anionic carboxylate group, thereby providing the final iodinated polyamino compound.

In various embodiments, the iodinated polyamino compounds comprise a polyamino moiety having a plurality (two, three, four, five, six, seven, eight, nine, ten or more)amino groups. For example, the polyamino moiety may comprises a plurality of (two, three, four, five, six, seven, eight, nine, ten or more) —(CH₂)ₓ—NH₂ groups where x is 0, 1, 2 3, 4, 5 or 6. In some of these embodiments, the polyamino moiety may comprises a plurality of —(CH₂)ₓ—NH₂ groups disposed along a polymeric moiety (defined here as a moiety comprising 2, 3, 4, 5, 6, 7, 8, 9, 10 or more monomer residues).

In some embodiments, the polyamino moiety of the iodinated polyamino compounds may correspond to a residue of a carboxyl-substituted polyamino compound (a compound comprising a carboxyl group and a plurality of amino groups). Examples of carboxyl-substituted polyamino compounds include peptides containing from 2 to 10 lysine and/or ornithine amino acid residues, including polylysines (e.g., dilysine, trilysine, tetralysine, pentalysine, etc.) and carboxyl-terminated polyamines such as carboxyl-terminated poly(allyl amine), carboxyl-terminated poly(vinyl amine), or carboxyl-terminated chitosan.

Commercially available examples of carboxyl-substituted polyamino compounds also include 16-amino-3-[2-[(4-aminobutyl) (3-aminopropyl)amino]-2-oxoethyl]-12-(3-aminopropyl)-6,9-bis(carboxymethyl)-11-oxo-3,6,9,12-tetraazahexadecanoic acid, L-ornithyl-L-ornithyl-L-ornithine, N²-[1-[N²-[N² (N-L-valyl-L-alanyl)-L-lysyl]-L-lysyl]-L-prolyl]-L-Lysine, L-Lysyl-L-tryptophyl-L-lysyl-L-lysine, N²,N⁵,N⁵-tris(3-aminopropyl)-L-ornithine, L-lysyl-L-omithyl-L-lysine, D-lysyl-D-lysyl-D-lysine, glycylglycyl-L-lysylglycylglycyl-L-lysine, N²-[NA-[N-[N-(N-glycylglycyl)glycyl|glycyl]-L-lysyl]-L-lysine, L-Lysyl-L-threonyl-L-lysyl-L-lysine, glycylglycyl-L-lysyl-L-lysylglycyl-L-cysteine, L-lysyl-L-arginyl-L-lysyl-L-lysine, L-arginyl-L-lysyl-L-lysyl-L-lysine, L-leucyl-L-lysyl-L-seryl-L-lysyl-L-lysine, L-alanyl-L-methionylglycyl-L-lysyl-L-lysyl-L-lysine, L-lysyl-L-lysyl-L-lysyl-L-arginyl-L-glutamine, L-seryl-L-isoleucyl-L-lysyl-L-lysyl-Llysyl-L-lysine, N² (N²-L-ornithyl-L-lysyl)-L-lysine, lysyllysyl-lysine, and L-lysyl-L-lysyl-L-lysyl-L-alanine.

Iodinated polyamino compounds may be formed by a reaction in which the carboxyl group of a carboxyl-substituted polyamino compound such as those described above (after protecting the amino groups of the carboxyl-substituted polyamino compound with a suitable protective agent, e.g., by reaction with di-tert-butyl decarbonate or another protective agent) is reacted with the amino group of an iodinated amino acid C₁-C₅-alkyl ester such as those described above, to form an amide bond between the two residues. A particular compound formed, for example, by amide coupling between tBoc-protected trilysine and diiodotyrosine methyl ester is shown here (after tBoc deprotection and hydrolysis of the methyl ester):

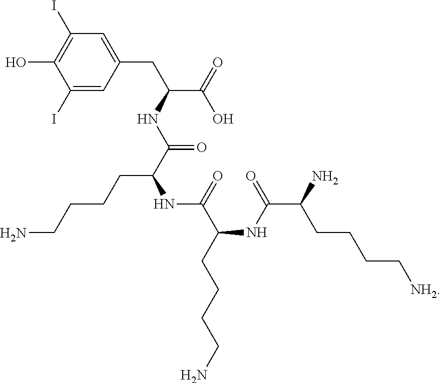 [SMILES: NCCCC[C@H](NC(=O)[C@H](CCCCN)NC(=O)[C@@H](N)CCCCN)C(=O)N[C@@H](Cc1cc(I)c(O)c(I)c1)C(=O)O]

Other examples of iodinated polyamino compounds include those formed from polyhydroxylated iodinated compounds by substituting hydroxyl groups of the polyhydroxylated iodinated compounds with amino groups (—NH₂ groups) by various suitable techniques. In one process, analogous to the first step shown in FIG. 4, hydroxyl groups of a polyhydroxylated iodinated compound are converted into methanesulfonate groups, followed by reaction with ammonia and treatment with hydrochloric acid to convert the methanesulfonate groups to amino groups (in ammonium salt form). As another example, hydroxyl groups of a polyhydroxylated iodinated compound are converted into methanesulfonate groups, followed by reaction with sodium amide, NaNH₂ (also known as sodium azanide) and treatment with hydrochloric acid to convert the methanesulfonate groups to amino groups (in ammonium salt form).

Using these and other techniques, a variety of iodinated polyamino compounds may be formed from iodinated polyhydroxylated compounds by amino substitution of all or a portion of the hydroxyl groups of the iodinated polyhydroxylated compounds. Such iodinated polyamino compounds may contain two, three, four, five, six, seven, eight, nine, ten, eleven, twelve or more amino groups.

Particular examples of iodinated polyamino compounds include the following, among others: iopromide, wherein at least a portion of the hydroxyl groups, specifically, four hydroxyl groups, have been substituted by amino groups, with the result being N,N′-Bis(2,3-diaminopropyl)-2,4,6-triiodo-5-(2-methoxyacetamido)-N-methylisophthalamide,

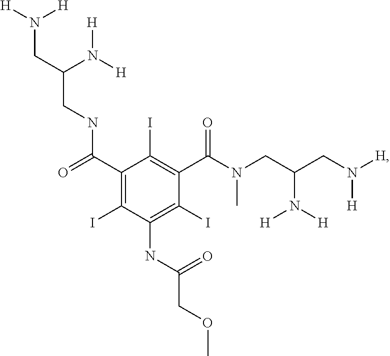

iopamidol, wherein at least a portion of the hydroxyl groups, specifically, five hydroxyl groups, have been substituted by amino groups, with the result being N,N′-bis(2-amino-1-(aminomethyl)ethyl)-2,4,6-triiodo-5-lactamidoisophthalamide

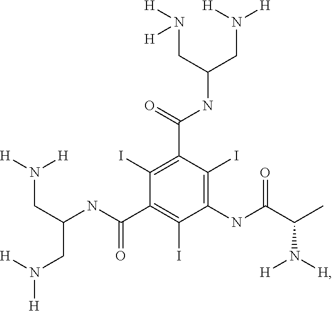

iomeprol, wherein at least a portion of the hydroxyl groups, specifically, five hydroxyl groups, have been substituted by amino groups, with the result being N,N′-bis(2,3-aminopropyl)-5-(2-amino-N-methylacetamido)-2,4,6-triiodoisophthalamide,

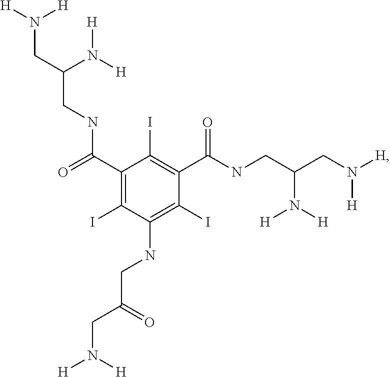

ioversol, wherein at least a portion of the hydroxyl groups, specifically, six hydroxyl groups, have been substituted by amino groups, with the result being N,N′-bis(2,3-diaminopropyl)-5-(2-amino-N-(2-amino) acetamido)-2,4,6-triiodoisophthalamide,

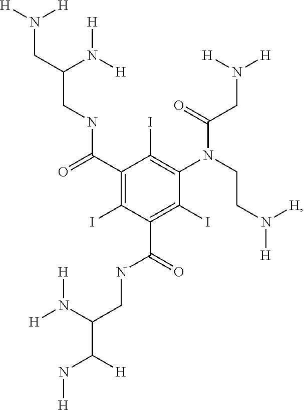

iobitridol, wherein at least a portion of the hydroxyl groups, specifically, six hydroxyl groups, have been substituted by amino groups, with the result being N,N′-bis(2,3-diaminopropyl)-5-((5-amino-2-(aminomethyl)-1-oxopropyl)amino)-2,4,6-triiodo-N,N′-dimethyl-1,3-benzenedicarboxamide,

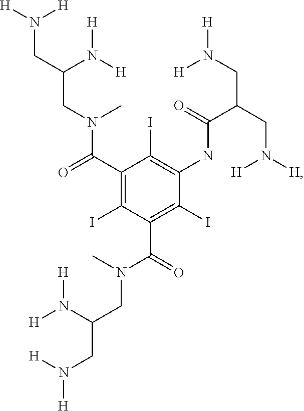

iodixanol, wherein at least a portion of the hydroxyl groups, specifically, eight out of nine hydroxyl groups, have been substituted by amino groups, with the result being 5,5′-((2-hydroxytrimethylene)bis(acetylimino)) bis(N,N′-bis(2,3-diaminopropyl)-2,4,6-triiodoisophthalamide,

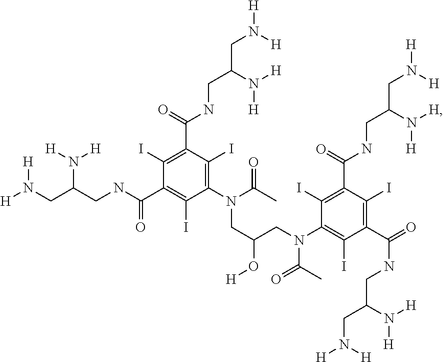

and iotrolan, wherein at least a portion of the hydroxyl groups, specifically, twelve hydroxyl groups, have been substituted by amino groups, with the result being 5,5′-((1,3-dioxo-1,3-propanediyl)bis(methylimino))bis(N,N′-bis(2,3-diamino-1-(aminomethyl) propyl)-2,4,6-triiodo-1,3-benzenedicarboxamide,

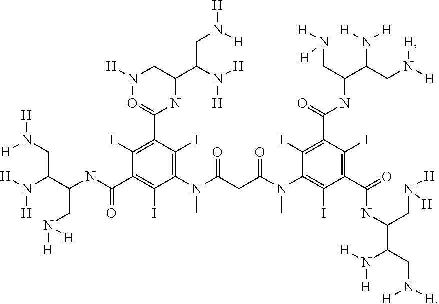

In some embodiments, the iodinated polyamino compound need not be formed from an iodinated polyhydroxylated compound. One example of such an iodinated polyamino compound is CA⁴⁺:

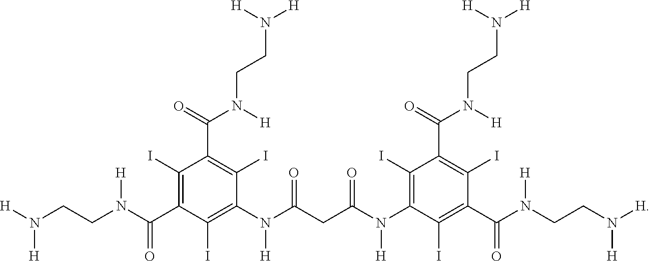

As previously noted, in some aspects, the present disclosure provides hydrogels that comprise a hydrolysis-resistant crosslinked reaction product of (a) a polyamino compound, such as those described above, and (b) a reactive polymer comprising reactive moieties that are reactive with amino groups of the polyamino compound, such as those described above.

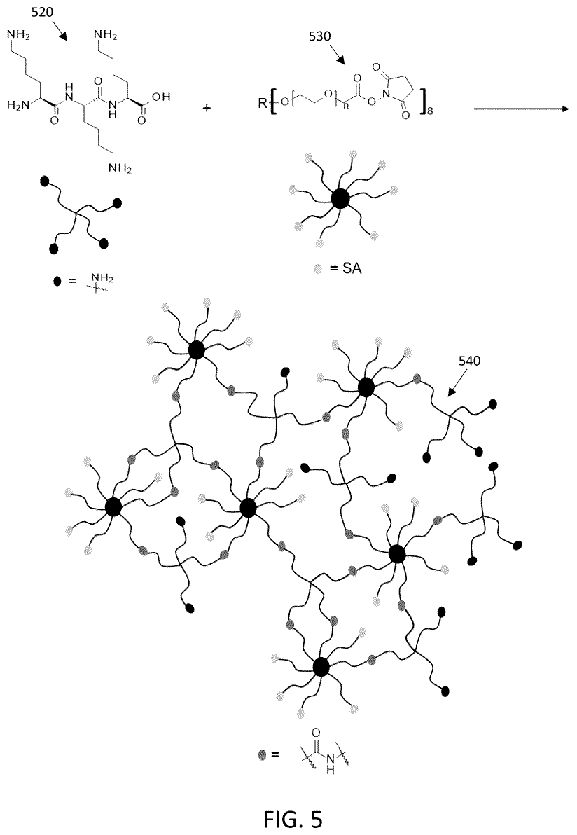

A specific embodiment is shown in FIG. 5, which schematically illustrates a crosslinking reaction between a polyamino compound, specifically, trilysine (520), and a reactive polymer having reactive moieties that comprise reactive cyclic imide ester groups, specifically, an eight-arm PEG with reactive succinimidyl carboxymethyl ester (SA) groups (530), having a core region that comprises a polyol residue, for example, a tripentaerythritol polyol residue or a hexaglycerol polyol residue, where R represents the polyol residue, and n ranges from 30 to 140. The reactive polymer (530) and the polyamino compound (520) are combined under conditions such that the succinimidyl ester groups of the reactive polymer (530) react with the amino groups of the polyamino compound (520) to form amide bonds accompanied by the release of N-hydroxysuccinimide, with the result being a crosslinked polymer network (540). The amide bond formation eliminates the ester group, with the result being an ester-free amide linkage such that the crosslinked polymer network (540) is resistant to hydrolysis. In certain embodiments, reaction between the succinimidyl ester groups and the amino groups is conducted at slightly basic pH (e.g., having a pH value ranging from 7.4 to 11) where the amino groups of the polyamino compound are deprotonated/neutrally charged and amide bond formation can occur spontaneously at body temperature.

Although not illustrated, in another specific embodiment, a polyamino compound as described herein may be reacted with a reactive multi-arm polymer having reactive moieties that comprise isocyanate groups, for example, an eight-arm PEG with reactive isocyanate groups having a core region that comprises a tripentaerythritol or hexaglycerol residue like that described above. The reactive multi-arm polymer and the polyamino compound may be combined under conditions such that the isocyanate groups of the reactive hydrophilic polymer react with the primary amine groups of polyamino compound to form urea bonds, thereby forming a crosslinked polymer network. In various embodiments, a non-aqueous solution of the reactive isocyanate multi-arm polymer is reacted with a non-aqueous solution of the polyamino compound to form the crosslinked polymer network. Examples of non-aqueous solutions include dimethyl sulfoxide (DMSO) solutions.

It will be appreciated that the crosslinking density of the hydrolysis-resistant crosslinked hydrogels described herein can be tuned, for example, (a) by varying the number of primary amine groups in the polyamino compound, (b) by varying the number of arms of the reactive multi-arm polymer, or (c) both.

In some embodiments, the hydrolysis-resistant crosslinked hydrogel is visible under fluoroscopy. The hydrolysis-resistant crosslinked hydrogel may have a radiopacity that is greater than 100 Hounsfield units (HU), beneficially ranging anywhere from 100 HU to 250 HU to 500 HU to 750 HU to 1000 HU or more (in other words, ranging between any two of the preceding numerical values), for example, when measured on bench-top micro CT systems such as XtremeCT from Scanco Medical (Wangen-Brüttisellen, Switzerland) or similar.

In some aspects of the present disclosure, a system is provided that comprises (a) a first composition that comprises a polyamino compound as described herein and (b) a second composition that comprises a reactive polymer comprising reactive moieties as described herein, wherein the system is configured to deliver the reactive polymer and the polyamino compound under conditions such that covalent crosslinks are formed between the reactive polymer and the polyamino compound.

For example, in the case where the reactive polymer is a reactive polymer comprising reactive isocyanate moieties, the first composition may be a first fluid composition comprising the polyamino compound and a suitable non-aqueous solvent such as DMSO, or the first composition may be a first dry composition that comprises the polyamino compound, to which a suitable non-aqueous solvent can be added to form a first fluid composition. In addition to the polyamino compound, the first composition may further comprise additional agents, including therapeutic agents, imaging agents, colorants, tonicity adjusting agents, suspension agents, wetting agents, and pH adjusting agents as described below. The second composition may be a second fluid composition comprising the reactive polymer comprising reactive isocyanate moieties and a suitable non-aqueous solvent such as DMSO, or the second composition may be a second dry composition that comprises the reactive polymer comprising reactive isocyanate moieties, to which a suitable non-aqueous solvent can be added to form a to form a second fluid composition. In addition to the reactive polymer, the second composition may further comprise additional agents, including therapeutic agents, imaging agents, colorants, tonicity adjusting agents, suspension agents, wetting agents, and pH adjusting agents as described below.

In the case where the reactive polymer is a reactive polymer comprising reactive electrophilic moieties, the first composition may be a first fluid composition comprising the polyamino compound or a first dry composition that comprises the polyamino compound, to which a suitable fluid such as water for injection, saline, etc. can be added to form a first fluid composition. In addition to the polyamino compound, the first composition may further comprise additional agents, including therapeutic agents, imaging agents, colorants, tonicity adjusting agents, suspension agents, wetting agents, and pH adjusting agents as described below. The second composition may be a second fluid composition comprising the reactive polymer comprising reactive electrophilic moieties or a second dry composition that comprises the reactive polymer comprising reactive electrophilic moieties, to which a suitable fluid such as water for injection, saline, etc. can be added to form a second fluid composition. In addition to the reactive polymer comprising reactive electrophilic moieties, the second composition may further comprise additional agents, including therapeutic agents, imaging agents, colorants, tonicity adjusting agents, suspension agents, wetting agents, and pH adjusting agents as described below.

In some embodiments, the system is configured to combine a first fluid composition comprising the polyamino compound with a second fluid comprising the reactive polymer. Upon mixing the first and second fluid compositions, the polyamino compound crosslinks with the reactive polymer, forming a crosslinked product. The first and second fluid compositions may be combined to form hydrolysis-resistant crosslinked hydrogels.

In some embodiments, the polyamino compound is initially combined with the reactive polymer under conditions where crosslinking between the reactive polymer and the polyamino compound is suppressed (e.g., an acidic pH in the case where the reactive polymer comprises reactive electrophilic moieties). Then, when crosslinking is desired, the conditions are changed such that crosslinking is increased (e.g., a change from an acidic pH to a basic pH, in some embodiments), leading to crosslinking between the polyamino compound and the reactive polymer, thereby forming a crosslinked product. The first and second fluid compositions may be combined to form hydrolysis-resistant crosslinked hydrogels.

In some of these embodiments, the system comprises (a) a first composition that comprises a polyamino compound as described hereinabove, (b) a second composition that comprises a reactive polymer comprising reactive electrophilic moieties as described hereinabove, and (c) a third composition, specifically, an accelerant composition, that contains an accelerant that is configured to accelerate a crosslinking reaction between the polyamino compound and the reactive polymer comprising reactive electrophilic moieties.

The first composition may be a first fluid composition comprising the polyamino compound that is buffered to an acidic pH or a first dry composition that comprises the polyamino compound, to which a suitable fluid such as water for injection, saline, an acidic buffer solution, etc. can be added to form a first fluid composition comprising the polyamino compound that is buffered to an acidic pH. In some embodiments, for example, the acidic buffering composition may comprise monobasic sodium phosphate, among other possibilities. The first fluid composition comprising the polyamino compound may have a pH ranging, for example, from about 3 to about 6.5, typically, from about 3 to about 5. In addition to the polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer comprising reactive electrophilic moieties or a second dry composition that comprises the reactive polymer comprising reactive electrophilic moieties from which a fluid composition is formed, for example, by the addition of a suitable fluid such as water for injection, saline, or the first fluid composition comprising the polyamino compound that is buffered to an acidic pH. In addition to the reactive polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the polyamino compound that is buffered to an acidic pH and the second composition comprises a dry composition that comprises the reactive polymer comprising reactive electrophilic moieties. The first composition may then be mixed with the second composition to provide a prepared fluid composition that is buffered to an acidic pH and comprises the polyamino compound and the reactive polymer. In a particular example, a syringe may be provided that contains the first fluid composition comprising the polyamino compound that is buffered to an acidic pH, and a vial may be provided that comprises the dry composition (e.g., a powder) that comprises the reactive polymer. The syringe may then be used to inject the first fluid composition into the vial containing the reactive polymer to form a prepared fluid composition that is buffered to an acidic pH and contains the polyamino compound and the reactive polymer, which can be withdrawn back into the syringe for administration.

The accelerant composition may be a fluid accelerant composition that is buffered to a basic pH or a dry composition that comprises a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 8.5 to about 12, typically, from about 9 to about 11. In addition to the above, the fluid accelerant composition may further comprise additional agents, including those described below.

A prepared fluid composition that is buffered to an acidic pH and comprises the polyamino compound and the reactive polymer comprising reactive electrophilic moieties as described above, and a fluid accelerant composition that is buffered to basic pH as described above, may be combined to form hydrolysis-resistant crosslinked hydrogels.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly(DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethene (BODIPY) analogs, among others, (e) imageable radioisotopes including 99mTc, 201Th, 51Cr, 67Ga, 68Ga, 111 In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a first fluid composition that comprises a polyamino compound as described herein and a second reservoir that contains a second fluid composition that comprises a reactive polymer as described herein, wherein the first and second fluid compositions form a crosslinked product upon mixing.

In some embodiments, the system may include a delivery device that comprises a first reservoir that contains a first fluid composition that comprises a polyamino compound as described herein and a reactive polymer comprising reactive electrophilic moieties as described herein and is buffered to an acidic pH, such as the prepared fluid composition previously described, and a second reservoir that contains second fluid composition, such as the fluid accelerant composition described herein.

In either case, during operation, the first fluid composition and the second fluid composition are dispensed from the first and second reservoirs and combined, whereupon the polyamino compound and the reactive polymer and crosslink with one another to form a hydrolysis-resistant crosslinked hydrogel.

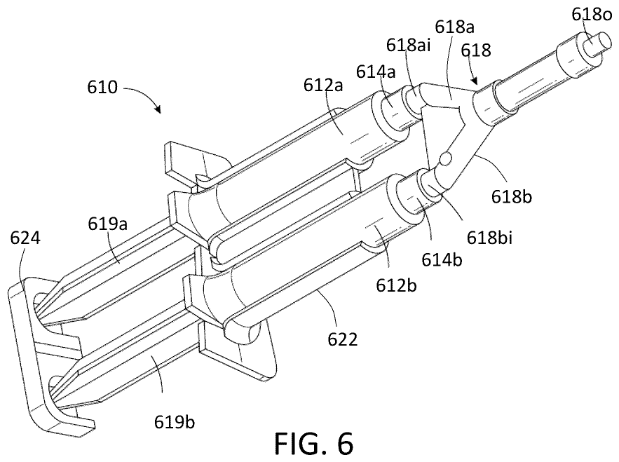

In particular embodiments, and with reference to FIG. 6, the system may include a delivery device 610 that comprises a double-barrel syringe, which includes a first barrel 612a having a first barrel outlet 614a, which first barrel contains a first fluid composition as described above, a first plunger 619a that is movable in the first barrel 612a, a second barrel 612b having a second barrel outlet 614b, which second barrel 612b contains a second fluid composition as described above, and a second plunger 619b that is movable in the second barrel 612b. In some embodiments, the device 610 may further comprise a mixing section 618 having a first mixing section inlet 618ai in fluid communication with the first barrel outlet 614a, a second mixing section inlet 618bi in fluid communication with the second barrel outlet 614b, and a mixing section outlet 6180. Also shown are a syringe holder 622 configured to hold the first and second syringe barrels 612a, 612b, in a fixed relationship and a plunger cap 624 configured to hold the first and second plungers 619a, 619b in a fixed relationship. In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a luer connector.

In some embodiments, the delivery device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a hydrolysis-resistant crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

In particular embodiments, the system may include (a) a preloaded first syringe filled with a polyamine solution containing a polyamino compound as described herein and is buffered to an acidic pH, for example, a trilysine solution that is buffered to a pH ranging from 3.8 to 4.2, (b) a vial that contains a powder of a reactive polymer comprising reactive electrophilic moieties as described herein, for example, an eight-arm PEG wherein the polymer arms contain terminal succinimidyl carboxymethyl ester groups, which reactive polymer may have a number average molecular weight ranging, for example, from 10000 to 20000 Daltons, (c) a preloaded second syringe containing a fluid accelerant composition that is buffered to a basic pH, for example, to a pH ranging from about 9.8 to 10.4. In use, the first syringe may be used to inject the polyamine solution into the vial to form a prepared fluid composition that is buffered to an acidic pH and contains the polyamino compound and the multi-arm polymer, which can be withdrawn back into the first syringe for administration. The prepared solution may contain, for example, from 0.1 to 1.0 wt % lysine and from 0.5 to 12 wt % eight-arm PEG. The system may further comprise a delivery device like that of FIG. 6, which may be loaded with the first syringe containing the prepared fluid composition and the second syringe containing the fluid accelerant composition and may be used to simultaneously inject and mix the prepared fluid composition and the fluid accelerant composition.

Regardless of the type of system that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

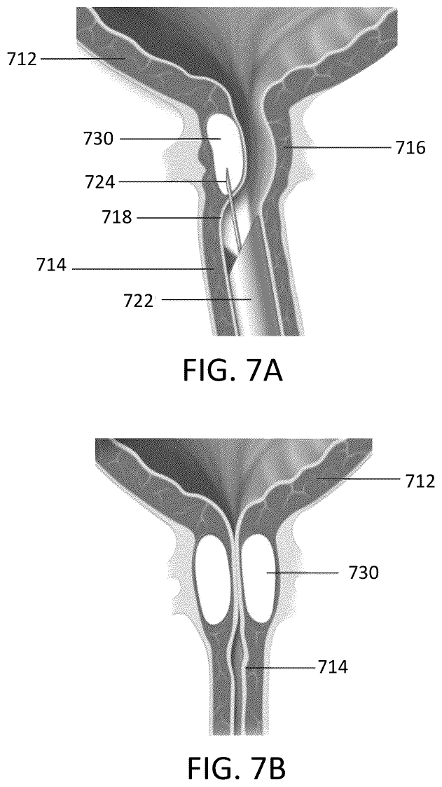

For example, in some embodiments, the first and second fluid compositions or a fluid admixture thereof can be injected as a bulking agent. For example, the first and second fluid compositions or a fluid admixture thereof may be injected to increase coaptation of a bodily sphincter, such as an anal sphincter for fecal incontinence or a urinary sphincter. FIGS. 7A and 7B illustrate a portion of the human anatomy that includes a lower portion of the bladder wall 712, an upper portion of the urethra 714, and the bladder neck 716, which a group of muscles that connect the bladder to the urethra. A fluid admixture of the first and second fluid compositions is injected by a transurethral route using a suitable delivery device such as that shown in FIG. 6, where a catheter tube (not shown) and needle 724 are attached to the mixing section outlet 6180 of the delivery device 610. In the embodiment shown, the injection is performed with the aid of a cystoscope 722 through the urethral wall 718 into the bladder neck 716, increasing coaptation of the urinary sphincter. After injection, the polyamino compound and the reactive polymer in the fluid admixture crosslink to form a hydrolysis-resistant hydrogel 730. In the case where the fluid admixture contains a non-aqueous solvent such as DMSO, the non-aqueous solvent will eventually be replaced by water from surrounding bodily fluids.

In other examples, the first and second fluid compositions or a fluid admixture thereof can be injected as a bulking agent into tissue around the ureteral orifices for the treatment of vesicoureteral reflux, the first and second fluid compositions or a fluid admixture thereof can be injected for tissue augmentation or regeneration, including cosmetic tissue augmentation, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a hydrolysis-resistant crosslinked hydrogel is ultimately formed at the administration location.

During and/or after administration, the compositions of the present disclosure can be imaged using a suitable imaging technique. Typically, the imaging techniques is an x-ray-based imaging technique, such as computerized tomography or x-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to occlude either a vas deferens or fallopian tube with a crosslinked product of the first and second fluid compositions for the control of reproductive health/family planning, a procedure to implant an embolic composition comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, injection for obstruction of the vas deferens, injection for obstruction of the fallopian tube, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

In some embodiments, the hydrolysis-resistant crosslinked hydrogels are formed ex vivo, in which case the hydrolysis-resistant crosslinked hydrogels may be in any desired form, including a slab, a cylinder, a coating, or a particle. In some embodiments, the hydrolysis-resistant crosslinked hydrogel is dried and then granulated into particles of suitable size. Granulating may be by any suitable process, for instance by grinding (including cryogrinding), homogenization, crushing, milling, pounding, pressing through a screen, or the like. Sieving or other known techniques can be used to classify and fractionate the particles. Hydrogel particles formed using the above and other techniques may varying widely in size, for example, having an average size ranging from 50 to 950 microns.

In addition to a hydrolysis-resistant crosslinked hydrogel that is formed ex vivo, hydrogel compositions in accordance with the present disclosure may be provided, which contain additional agents, including therapeutic agents, imaging agents, colorants, tonicity adjusting agents, suspension agents, wetting agents, and pH adjusting agents as described above.

In various embodiments, kits are provided that include one or more delivery devices for delivering such hydrogel compositions to a subject. Such systems may include one or more of the following: a syringe barrel, which may or may not contain a hydrogel composition; a vial, which may or may not contain a hydrogel composition; a needle; a flexible tube (e.g., adapted to fluidly connect the needle to the syringe); and an injectable liquid such as water for injection, normal saline or phosphate buffered saline. Whether supplied in a syringe, vial, or other reservoir, the hydrogel composition may be provided in dry form (e.g., powder form) or in a form that is ready for injection, such as an injectable hydrogel form (e.g., a suspension of hydrogel particles).

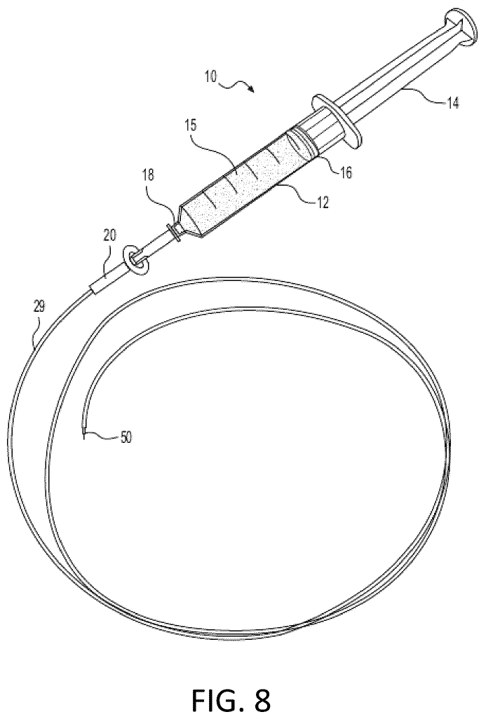

FIG. 8 illustrates a syringe 10 providing a reservoir for a hydrogel composition in accordance with the present disclosure. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a hydrogel composition 15 for injection through the needle 50.

The hydrogel compositions described herein (e.g., a suspension of hydrogel particles, which may also optionally contain additional agents described above) can be used for a number of purposes.

For example, hydrogel compositions can be injected to provide spacing between tissues, hydrogel compositions can be injected (e.g., in the form of blebs) to provide fiducial markers, hydrogel compositions can be injected for tissue augmentation or regeneration, hydrogel compositions can be injected as a filler or replacement for soft tissue, hydrogel compositions can be injected for tissue bulking, hydrogel compositions can be injected for occlusion of lumens, hydrogel compositions can be injected to provide mechanical support for compromised tissue, hydrogel compositions be injected as a scaffold, and/or hydrogel compositions can be injected as a carrier of therapeutic agents in the treatment of diseases and cancers and the repair and regeneration of tissue, among other uses.

During or after administration, the hydrogel compositions of the present disclosure can be imaged using a suitable imaging technique.

As seen from the above, the hydrogel compositions of the present disclosure may be used in a variety of medical procedures, including the following, among others: a procedure to implant a fiducial marker comprising a hydrogel, a procedure to implant a tissue regeneration scaffold comprising a hydrogel, a procedure to implant a tissue support comprising a hydrogel, a procedure to implant a tissue bulking agent comprising a hydrogel, a procedure to occlude a lumen, a procedure to implant a therapeutic-agent-containing depot comprising a hydrogel, a tissue augmentation procedure comprising implanting a hydrogel, a procedure to introduce a hydrogel between a first tissue and a second tissue to space the first tissue from the second tissue, among others.

The hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, injection to increase coaptation of a bodily sphincter such as an anal sphincter for or a urinary sphincter, injection for vas deferens occlusion, injection for fallopian tube occlusion, injection for seminal vessel occlusion, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Hydrogel compositions in accordance with the present disclosure include lubricious compositions for medical applications, compositions for therapeutic agent release (e.g., by including one or more therapeutic agents in a matrix of the hydrogel), and implants (which may be formed ex vivo or in vivo) (e.g., compositions for use as tissue markers, compositions that act as spacers to reduce side effects of off-target radiation therapy, cosmetic compositions, etc.).

It should be understood that this disclosure is, in many respects, only illustrative and that changes may be made in details without exceeding the scope of the disclosure. This may include, to the extent that it is appropriate, the use of any of the features of one embodiment being used in other embodiments.

1. A system for forming a hydrogel that comprises (i) a reactive multi-arm polymer that comprises three or more polymer arms linked to a core region, each arm comprising a polyether segment and a reactive moiety comprising a cyclic imide carboxy-C₁-C₅-alkyl ester end group or each arm comprising a polyether segment and a reactive moiety comprising a C₂-C₆-isocyanoalkyl end group and (ii) a polyamino compound comprising at least two amino (—NH₂) groups, wherein the reactive multi-arm polymer and the polyamino compound react to form a crosslinked hydrogel that does not contain ester groups and has long-term stability in vivo.

2. The system of claim 1, wherein the reactive moiety is a cyclic imide carboxy-C₁-C₅-alkyl ester group or a C₂-C₆-isocyanoalkyl group and wherein the reactive moiety is directly bonded to the polyether segment.

3. The system of claim 1, wherein the reactive multi-arm polymer is formed from a multi-arm polymer that comprises three or more polymer arms linked to a core region, each arm comprising a polyether segment and a C₂-C₆-hydroxyalkyl end group.

4. The system of claim 3, wherein the reactive multi-arm polymer is formed from a process that comprises oxidizing the C₂-C₆-hydroxyalkyl end group to form a C₂-C₆-carboxyalkyl end group and reacting the C₂-C₆-carboxyalkyl end group with a N-hydroxy cyclic imide compound in an ester coupling reaction to form the cyclic imide carboxy-C₁-C₅-alkyl ester end group.

5. The system of claim 1, wherein the polyether segment is a polyethylene oxide segment and the cyclic imide carboxy-C₁-C₅-alkyl ester end group is a cyclic imide carboxymethyl ester end group.

6. The system of claim 1, wherein the cyclic imide carboxy-C₁-C₅-alkyl ester end group is a succinimide carboxymethyl ester end group.

7. The system of claim 3, wherein the reactive multi-arm polymer is formed from a process that comprises converting the C₂-C₆-hydroxyalkyl end group to a C₂-C₆-aminoalkyl end group and reacting the C₂-C₆-aminoalkyl end group with phosgene to form the C₂-C₆-isocyanoalkyl end group.

8. The system of claim 1, wherein the polyether segment is a polyethylene oxide segment and the C₂-C₆-isocyanoalkyl end group is an isocyanoethyl end group.

9. The system of claim 1, wherein each of the polyether segments contains between 10 and 1000 monomer residues.

10. The system of claim 1, wherein the core region comprises a polyol residue.

11. The system of claim 1, wherein the core region is an iodinated core region.

12. The system of claim 1, wherein the polyamino compound comprises a plurality of basic amino acid residues.

13. The system of claim 1, wherein the polyamino compound is an iodinated polyamino compound.

14. The system of claim 1, comprising a first composition that comprises the polyamino compound in a first container and a second composition that comprises the reactive multi-arm polymer in a second container.

15. The system of claim 1, further comprising a delivery device.

16. A hydrolysis-resistant crosslinked hydrogel produced by covalent crosslinking between (i) a reactive multi-arm polymer that comprises three or more polymer arms linked to a core region, each arm comprising a polyether segment and a reactive moiety comprising a cyclic imide carboxy-C₁-C₅-alkyl ester end group or each arm comprising a polyether segment and a reactive moiety comprising a C₂-C₆-isocyanoalkyl end group and (ii) a polyamino compound comprising at least two amino (—NH₂) groups, wherein the reactive multi-arm polymer and the polyamino compound react to form a crosslinked hydrogel that does not contain ester groups and has long-term stability in vivo.

17. The hydrolysis-resistant crosslinked hydrogel of claim 16, wherein the hydrolysis-resistant crosslinked hydrogel has a radiopacity that is greater than 100 Hounsfield units (HU).

18. A method of treatment comprising administering to a subject a mixture that comprises (i) a reactive multi-arm polymer that comprises three or more polymer arms linked to a core region, each arm comprising a polyether segment and a reactive moiety comprising a cyclic imide carboxy-C₁-C₅-alkyl ester end group or each arm comprising a polyether segment and a reactive moiety comprising a C₂-C₆-isocyanoalkyl end group and (ii) a polyamino compound comprising at least two amino (—NH₂) groups, wherein the mixture is administered under conditions such that the polyamino compound and the reactive multi-arm polymer react to form a crosslinked hydrogel after administration and wherein the crosslinked hydrogel does not contain esters and has long-term stability in vivo.

19. The method of claim 18, wherein the method comprises administering to the subject (a) a first fluid composition that comprises the polyamino compound and the reactive multi-arm polymer, wherein the reactive moiety comprises the cyclic imide carboxy-C₁-C₆-alkyl ester end group, and (b) a second fluid composition that comprises an accelerant that accelerates reaction of the polyamino compound and the reactive multi-arm polymer.

20. The method of claim 18, wherein the method comprises administering to the subject (a) a first fluid composition that comprises the polyamino compound and a non-aqueous solvent and (b) a second fluid composition comprising the reactive multi-arm polymer and a non-aqueous solvent, wherein the reactive moiety comprises the C₂-C₆-isocyanoalkyl end group.
